(19) United States
(12) Patent Application Publication
(10) Pub. No.: US 2025/0059493 A1
(43) Pub. Date: Feb. 20, 2025

(54) THREE-DIMENSIONAL POROUS DEVICE FOR PURIFYING BIOLOGICAL MATERIALS

(71) Applicant: The Regents of the University of Michigan, Ann Arbor, MI (US)

(72) Inventors: Sunitha Nagrath, Ann Arbor, MI (US); Yoon-Tae Kang, Ann Arbor, MI (US)

(21) Appl. No.: 18/721,827

(22) PCT Filed: Dec. 22, 2022
(86) PCT No.: PCT/US22/82252

§ 371 Date: Jun. 19, 2024

Related U.S. Application Data
(60) Provisional application No. 63/292,904, filed on Dec. 22, 2021.

(51) Int. Cl. C12M 1/00; C09D 183/04; C12M 1/12

(52) U.S. Cl. CPC C12M 47/06; C09D 183/04; C12M 25/14

(57) ABSTRACT

The present disclosure provides porous three dimensional polymeric devices useful for purifying and/or isolating a target cell-derived component (e.g., extracellular vesicles), and methods of making and systems or kits comprising thereof. Wherein the three-dimensional device is a cube that comprises a polymer, and wherein the polymer comprises polydimethylsiloxane (PDMS).

### CROSS-REFERENCE TO RELATED APPLICATIONS

This application claims the benefit of U.S. Provisional Application No. 63/292,904, filed Dec. 22, 2021, the content of which is herein incorporated by reference in its entirety.

### FIELD

The present disclosure provides method for purifying or isolating a target cell-derived component (e.g., extracellular vesicles) using porous three-dimensional polymeric devices, methods of making porous three-dimensional polymeric devices, and systems or kits comprising the porous three-dimensional polymeric devices.

### BACKGROUND

Extracellular vesicles (EVs) are considered to be a promising cancer biomarker that circulates in most biological samples. The primary function of EVs is to facilitate cell-cell communication, and their innate information from their progenitor cells makes them the ideal biomarkers for early cancer diagnoses and metastasis. The presence of EVs in various biological fluids secreted by the body can be used for isolating and analyzing the exosomes for cancer diagnoses in the form of liquid biopsy. The separation of cancer-specific exosomes efficiently is limited due to the complexity of the biological sample's content. This results in many of the current methods not being able to effectively isolate specific exosomes of interest

### SUMMARY

Disclosed herein are methods for purifying or isolating a target cell-derived component. The methods comprise at least one or all of: incubating a sample with at least one porous three-dimensional device, wherein each device comprises a polymer-based surface conjugated with a binding agent for the target cell-derived component; removing unbound sample; eluting the target cell-derived component with an elution buffer composition configured to disrupt target cell-derived component and binding agent interaction. In some embodiments, the methods do not comprise centrifugation.

In some embodiments, the at least one porous three-dimensional device is a cube, cuboid, pyramid, prism, or polyhedron. In some embodiments, the at least one porous three-dimensional device comprises pores from 100 to 450 microns. In some embodiments, the at least one porous three-dimensional device has a maximum accommodated aqueous volume of at least 0.5 mLs. In some embodiments, the polymer comprises polydimethylsiloxane (PDMS).

In some embodiments, the incubating comprises fully or partially submerging the at least one porous three-dimensional device in the sample for a period of time. In some embodiments, the incubating further comprises agitating the fully or partially submerged at least one porous three-dimensional device in the sample. In some embodiments, the incubating further comprises adding binding composition which facilitates target cell-derived component and binding agent interaction.

In some embodiments, the sample is a biological sample. In some embodiments, the biological sample is obtained from a subject. In some embodiments, the subject has or is suspected of having a disease or disorder. In some embodiments, the sample has a volume of at least 1 mL (e.g., at least 50 mLs).

In some embodiments, the target cell-derived component comprises cell fragments. In some embodiments, the target cell-derived component comprises a biomolecule.

In some embodiments, the target cell-derived component comprises extracellular vesicles. In some embodiments, the target cell-derived component comprises exosomes. In some embodiments, the target cell-derived component comprises cancer cell-derived exosomes. In some embodiments, the binding agent comprises annexin V. In some embodiments, the binding composition comprises calcium. In some embodiments, the at least one porous three-dimensional device has a maximum exosome binding capacity of greater than 2×10⁸ exosomes per mL.

In some embodiments, the methods further comprise analyzing the target cell-derived component. In some embodiments, the analyzing comprises conducting a biological assay on the target cell-derived component.

Further disclosed herein are three-dimensional porous devices comprising a polymer-based surface conjugated with a binding agent and methods of fabricating thereof. In some embodiment, the device is a porous cube.

Additionally disclosed herein are systems or kits comprising a porous three-dimensional device as disclosed herein or components necessary for fabricating a porous three-dimensional device as disclosed herein.

Other aspects and embodiments of the disclosure will be apparent in light of the following detailed description and accompanying figures.

### BRIEF DESCRIPTION OF THE DRAWINGS

FIG. 1 is a schematic of an exemplary ^{Porous}ExoChip (ExoSponge) device, as disclosed herein, comprising exosome capturing molecules (such as Annexin V, anti-CD63, and the like) functionalization to confer high affinity for extracellular vesicles in a biological sample.

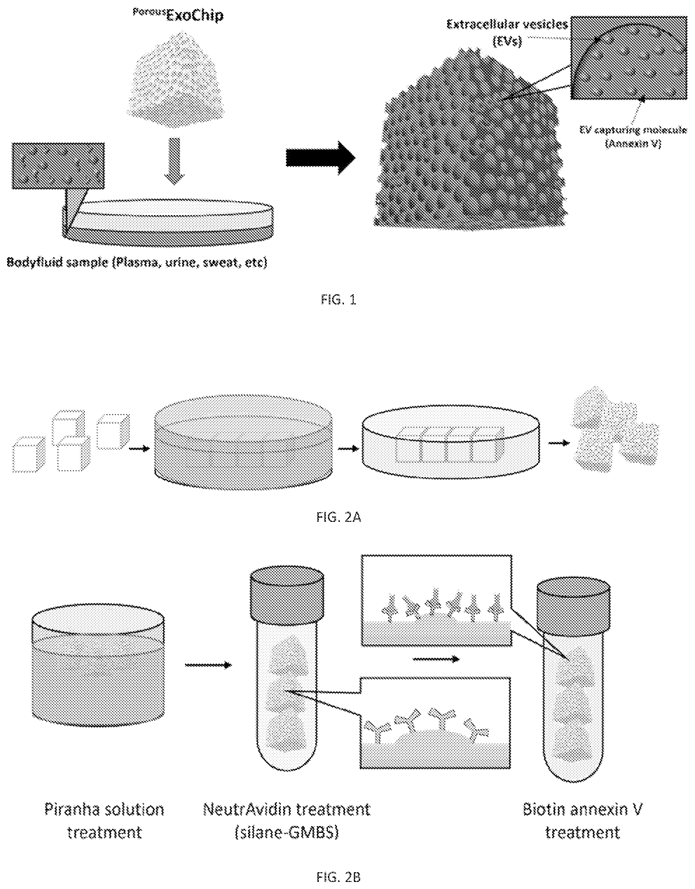

FIGS. 2A and 2B are schematics of the fabrication and surface modification of porous PDMS cubes to isolate circulating biomarkers. FIG. 2A shows creation of a device as disclosed herein using a sugar cube scaffold. FIG. 2B shows functionalization by piranha solution oxidation followed by Neutravidin Annexin V treatment.

FIGS. 3A-3C show evaluation of surface functionalization using biotinylated fluorescence dye. FIG. 3A is a graph of the quantitative analysis of fluorescence intensities between functionalized device and control device. FIGS. 3B and 3C are fluorescence images of the functionalized device (FIG. 3B) and control device (FIG. 3C).

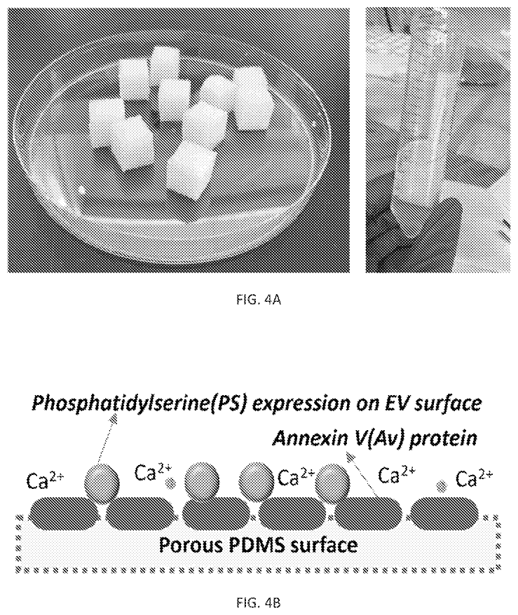

FIGS. 4A and 4B show ^{Porous}ExoChip surface modification. FIG. 4A shows making the surface hydrophilic for aqueous absorption. FIG. 4B shows annexin V conjugated binding interaction with phosphatidylserine protein.

FIGS. 5A-5C show porous microstructure analysis. FIG. SA are images of the porous microstructure which allows for high surface interaction. FIG. 5B shows hydrophilic aqueous absorption. FIG. 5C is a graph of the distribution of PDMS pore sizes.

FIGS. 6A-6C show exosome isolation using ^{Porous}ExoChip. FIG. 6A is a graph of incubation time optimization showing increasing EV isolation with increasing time. FIG. 6B is a graph of exosome isolation performance of an exemplary device and method as disclosed herein in comparison with traditional ultracentrifugation exosome isolation. The ^{Porous}ExoChip had double the exosome isolation. FIG. 6C is a graph comparing exosomal isolation between ^{Porous}ExoChip and ultracentrifugation along with their purity of the recovered exosomes.

### DETAILED DESCRIPTION

Disclosed herein are devices comprising a porous structure (e.g., PDMS) and functionalized to provide an inexpensive and efficient means of isolating target cell-derived components (e.g., extracellular vesicles (EVs)). Previous EV isolation methods such as ultracentrifugation require expensive equipment operated in a laboratory and are also limited by a small sample volume. Microfluidic devices are limited in use for EV isolation due to their incapability of handling larger samples volumes, such as those from bulk body fluid samples. The disclosed devices demonstrated capture results of which far exceed ultracentrifugation and possess the ability to analyze large volume samples. In some embodiments, the device is created using a sugar scaffolding for the polymer mold, followed by oxidation treatment to make it compatible with aqueous biological samples. After functionalization with a specific binding agent, the device surface comprises a high affinity for the target cell-derived components.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0. the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

A “biomarker” includes a biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, a cell fragment, an exosome, and a small molecule, that is present in a biological sample and that may be isolated from, or measured in, the biological sample. Furthermore, a biomarker may be the entire intact molecule, or a portion thereof that may be partially functional or recognized, for example, by an antibody or other specific binding protein. A biomarker may be associated with a given state of a subject, such as a particular stage of disease. In some embodiments, the biomarker is a cancer biomarker (e.g., circulating tumor DNA, protein biomarkers (e.g., prostate specific antigen, alpha-fetoprotein, carcinoembryonic antigen). A measurable aspect of a biomarker may include, for example, the presence, absence, or concentration of the biomarker in the biological sample from the subject and/or relative changes of any of the measurable aspects compared to a standard (e.g., internal or from a healthy subject). The measurable aspect may also be a ratio of two or more measurable aspects of two or more biomarkers. Biomarker, as used herein, also encompasses a biomarker profile comprising measurable aspects of two or more individual biomarkers. The two or more individual biomarkers may be from the same or different classes of biomarkers such as, for example, a nucleic acid and a carbohydrate, or may measure the same or different measurable aspect such as, for example, absence of one biomarker and concentration of another. A biomarker profile may comprise any number of individual biomarkers or features thereof. In another embodiment, the biomarker profile comprises at least one measurable aspect of at least one internal standard. Methods of identifying and quantifying biomarkers are well known in the art and include histological and molecular methods such as enzyme-linked immunosorbent assays (ELISA) and other immunoassays, gel electrophoresis protein and DNA arrays, mass spectrometry, colorimetric assays, electrochemical assays, and fluorescence methods.

The term “biomolecule(s)” as used herein refers to molecules typically produced by living organisms. These molecules may include peptides, proteins, glycoproteins, nucleic acids, fatty acids or lipids, and sugars, that exist extracellularly or intracellularly.

The terms “purifying” or “isolating” or “separating” are used to mean the removal, whether completely or partially, of at least one impurity from a mixture containing the target cell-derived component, which thereby improves the level of purity of the target cell-derived component (e.g., by decreasing the amount or percentage of impurity(ies) in the composition).

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Purifying a Target Cell-Derived Component

The present disclosure provides methods for purifying or isolating a target cell-derived component. The methods may comprise incubating a sample with at least one porous three-dimensional device, wherein each device comprises a polymer-based surface conjugated with a binding agent for the target cell-derived component, removing unbound sample, and eluting the target cell-derived component with an elution buffer composition configured to disrupt target cell-derived component and binding agent interaction. In some embodiments, the methods do not comprise centrifugation.

### a) Porous Three-Dimensional Device

The present disclosure further provides porous three-dimensional devices for use in purifying or isolating a target cell-derived component. The device may be any three-dimensional shape, including but not limited to, a cube, cuboid, pyramid, prism, or polyhedron. In some embodiments, the device is a cube.

The porous three-dimensional device may be variable in size but is generally between 0.25 cm and 5 cm in any one or all dimension (e.g., length, width, height). In some embodiments the porous three-dimensional device is about 0.5 cm, about 0.75 cm, about 1.0 cm, about 1.25 cm, about 1.5 cm, about 1.75 cm, about 2.0 cm, about 2.5 cm, about 3.0 cm, about 3.5 cm, about 4.0 cm, or about 4.5 cm in any one dimension. In some embodiments. the porous three-dimensional device is a cube between 0.25 cm and 5 cm in all dimensions.

In some embodiments, the device comprises pores ranging from about 100 microns to about 450 microns. In some embodiments, the average pore size over the whole of the device is between 200 and 300 microns.

Due to the porosity, the three-dimensional device can accommodate or hold an aqueous volume of sample. In some embodiments, the device has a maximum accommodated aqueous volume of at least 0.5 mLs. In some embodiments, the device has a maximum accommodated aqueous volume of at least 0.6 mLs., at least 0.7 mLs., at least 0.8 mLs., at least 0.9 mLs., at least 1.0 mLs., at least 1.2 mLs., at least 1.5 mLs., or more. Accordingly, the three-dimensional device itself have a variable volume. In some embodiments, the volume is greater than 1.0 cm³, greater than 1.25 cm³, greater than 1.5 cm³, greater than 1.75 cm³, greater than 2.0 cm³, greater than 2.25 cm³, greater than 2.5 cm³, greater than 3.0 cm³, or larger.

The three-dimensional devices comprise a polymer-based surface. Suitable polymers for use in the devices include elastomers (e.g., thermosets or thermoplastics). The polymer may be selected from the group consisting of perfluoropolyether (PFPE), polydimethylsiloxane (PDMS), poly(tetramethylene oxide), poly(ethylene oxide), poly(oxetanes), polyisoprene, polybutadiene, fluoroolefin-based fluoroelastomers, and the like. In some embodiments, the polymer comprises polydimethylsiloxane (PDMS), also known as dimethylpolysiloxane or dimethicone.

The polymer-based surface is conjugated with or comprises a binding agent for the target cell-derived component. The nature of the binding agent will be dependent on the target cell-derived component. “Binding agent” is used herein to refer to a species (e.g., protein, nucleic acid, carbohydrate) that binds to and forms a complex with the target cell-derived component. The binding agent specifically binds the target cell-derived component. Binding agents include antibodies, as well as antigen-binding fragments thereof and other various forms and derivatives thereof as are known in the art, and other molecules comprising one or more antigen-binding domains that bind to an antigen molecule or a particular site (epitope) on the antigen molecule. In addition to antigen and antibody specific binding pairs, other specific binding agents can include biotin and avidin (or streptavidin), carbohydrates and lectins, complementary nucleotide sequences, effector and receptor molecules, cofactors and enzymes, enzymes and enzyme inhibitors, and the like.

Thus, the disclosure provides a three-dimensional porous device comprising a polymer-based surface conjugated with a binding agent. Descriptions provided above for the three-dimensional porous device for use in the provided methods (e.g., three-dimensional shape, polymer, and binding agent) are also suitable with the disclosed device.

In some embodiments, the device is a porous cube. In some embodiments, the polymer-based surface comprises PDMS. In some embodiments, the PDMS is oxidized to be hydrophilic.

In some embodiments, the device has a maximum accommodated aqueous volume of 1.0-2.0 mLs (e.g., about 1.0 mL, about 1.2 mL, about 1.5 mL, about 1.7 mL, or about 2.0 mL).

Also provided herein are methods for fabricating a porous three-dimensional device. The methods comprise providing a three-dimensional porous scaffold, wherein the scaffold is removable, covering the three-dimensional porous substrate with a polymer or polymer precursor, incubating the covered porous substrate, curing the polymer or polymer precursor, and removing the scaffold.

The three-dimensional porous scaffold may be any shape as described above, including but not limited to, a cube, cuboid, pyramid, prism, or polyhedron. In some embodiments, the scaffold is a cube.

The scaffold is removable by some mechanism which does not disrupt the final device structure or three-dimensional porosity. For example, the scaffold may be dissolvable in various solvents or enzymatically digestible. In some embodiments, the scaffold comprises carbohydrates which will dissolve in various solvent systems (e.g., water, buffer, alcohols) at various temperatures and conditions. In some embodiments, the scaffold is a sugar cube.

As described above for the disclosed methods, the polymer or polymer precursor for use in the methods of fabricating include elastomers (e.g., thermosets or thermoplastics). The polymer may be selected from the group consisting of perfluoropolyether (PFPE), polydimethylsiloxane (PDMS), poly(tetramethylene oxide), poly(ethylene oxide), poly(oxetanes), polyisoprene, polybutadiene, fluoroolefin-based fluoroelastomers, and the like. In some embodiments, the polymer comprises polydimethylsiloxane (PDMS), also known as dimethylpolysiloxane or dimethicone.

The amount of polymer necessary to cover the entire scaffold will be dependent on the nature of the scaffold. Preferably, an excess of polymer is used to ensure a consistent coat around the entire scaffold, both interior pores and exterior surface.

In some embodiments, the polymer further comprises a curing or crosslinking agent. Based on the selected polymer, a curing agent may be necessary to reach the final product, but some polymers are self-curing. The curing agent is any substance which participates in the reaction between the prepolymer and polymer to achieve sufficient polymerization. Common curing agents include polyaziridine, carbodiimide, polyisocyanates, and silanes.

In some embodiments, the polymers may be hydrophobic such that the methods further comprise rendering the polymer surface hydrophilic. In some embodiments, the rendering the polymer surface hydrophilic comprises oxidation of the polymer surface. The oxidation may comprise O₂ plasma treatment and/or solution based methods utilizing strong acids and peroxides.

In some embodiments, the methods comprise functionalization of the polymer with a binding agent. Functionalization or conjugation of the binding agent may be accomplished by numerous methods known in the art, including, for example, avidin-streptavidin, biotin, cyanogen bromide coupling, and/or the use of a linker. The binding agent can also be directly bound to the polymer using coupling agents such as bifunctional reagents or can be indirectly bound. Preferred methods are based upon the desired application, binding agent, sample, or binding conditions necessary for the target cell-derived component and binding agent interaction. Descriptions of the types of binding agents provided above are suitable for use here.

The disclosed devices or devices fabricated by the methods disclosed herein are equally suitable for use in the methods for purifying and isolating a target cell-derived components as described herein.

### b) Incubating and Eluting

The mixture of the sample and the porous three-dimensional device(s) is then incubated. In some embodiments, the incubating comprises fully or partially submerging the at least one device in the sample for a period of time.

Based on the volume of sample of the concentration of target cell-derived component, a suitable number of devices can be used. Thus, the method may further comprise selecting, based on sample volume and target cell-derived component concentration. the number of devices necessary to efficiently isolate or purify the desired quantity of cell-derived component concentration and the container size necessary for the incubation. In some embodiments, two or more devices are used for larger sample sizes and scaled to larger containers (e.g., e.g., 5 devices for 10 mL in a 20 mL or larger tube or flask; 10 devices for 20 mL in a 50 mL or larger tube or flask; etc.)

The incubation can be at any temperature with any degree of heating or cooling, including no heating or cooling, for example at 5° C., at 20° C. or ambient room temperature, or at 37° C. The incubation times can vary and are in no way limiting. For example, incubation can be anywhere between 10 minutes to overnight (e.g., 16 hours). The incubation can be with or without agitation, and the agitation during the incubation period can be constant or intermittent.

In some embodiments, the incubating further comprising adding binding composition which facilitates the target cell-derived component and binding agent interaction. For example, in some embodiments, the binding composition provides a cofactor, pH or conductivity necessary for the target cell-derived component and binding agent interaction.

Following the incubation, the resulting unbound sample can be removed from the device(s). Once the unbound sample is removed, the device(s) may be washed with an appropriate buffer or the binding composition that does not disrupt the target cell-derived component and binding agent interaction but removes non-specific interactions of non-target sample components with the devices or binding agent.

The target cell-derived component can be removed from the device with an elution buffer compositions configured to disrupt the cell-derived component and binding agent interaction. For example, the elution buffer may have an increased concentration of salt, a non-physiological pH (e.g., acidic or basic), a chaotrope or denaturing agent, a chelating agent, or a competitive binding agent.

### c) Sample

As used herein, the term “sample” is used in its broadest sense. In one sense, it is meant to include a specimen obtained from any source, including biological and environmental samples. Biological samples may be obtained from animals (including humans) and encompass fluids, solids, and/or tissues. Such examples are not however to be construed as limiting the sample types. In some embodiments, the sample is a fluid sample such as a liquid sample. Examples of liquid samples suitable for use with the devices disclosed herein include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. Liquid samples can be made from solid, semisolid, or highly viscous materials, such as fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation. Samples can comprise biological materials, such as cells, microbes, organelles, and biochemical complexes.

The biological sample may be obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like. In some embodiments, the sample is blood or blood products. Blood products are any therapeutic substance prepared from human blood. This includes whole blood; blood components (e.g., red blood cell concentrates or suspensions; platelets produced from whole blood or via apheresis; plasma; serum and cryoprecipitate); and plasma derivatives (e.g., coagulation factor concentrates).

In some embodiments, the sample is a biological sample obtained from a subject having or suspected of having a disease or disorder.

The sample can be obtained from the subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like. The samples may be freshly collected samples or from samples that have been stored frozen or refrigerated.

The methods are suitable for any volume of sample. Advantageously, large samples can be effectively processed using the disclosed devices and methods. In some embodiments, the sample has a liquid volume, either as obtained or following processing, of at least 1 mL (e.g., at least 5 mLs, at least 10 mLs, at least 20 mLs, at least 30 mLs, at least 40 mLs, at least 50 mLs, at least 60 mLs, at least 70 mLs, at least 100 mLs, or more). In some embodiments, the sample has a liquid volume of at least 50 mLs.

### d) Target Cell-Derived Component

The device facilitates retention of the target cell-derived component bound by the binding agent whereas other sample components remain in solution. The cell-derived component may be any component of a cell or derived from a cell or fragments of a cell. The cell-derived component may comprise any biomolecule or structure from or derived from a cell, including, but not limited to, macromolecules such as proteins and nucleic acids, biomolecular complexes such as a ribosome, structures such as membranes and organelles, and extracellular components, such as extracellular vesicles.

In some embodiments, the target cell-derived component is an extracellular vesicle. In some embodiments, the target cell-derived component is an exosome.

A unified vesicle nomenclature and classification system utilizing broadly accepted definitions has been elusive in the field. The term “extracellular vesicles,” as used herein, refers to a lipid membrane particles having a diameter (or largest dimension where the particles is not spheroid) of between about 30 nm and 10,000 nm. Extracellular vesicles encompass exosomes, ectosomes, microvesicles, microparticles, prostasomes, tolerosomes (which induce immunological tolerance to dietary antigens), apoptotic bodies (released by apoptotic cells), and nanovesicles. The term “exosome,” as used herein, refers to a membranous particle having a diameter (or largest dimension where the particles is not spheroid) of between about 30 nm and 150 nm, wherein at least part of the membrane of the exosomes is directly obtained or derived from a cell. Most commonly, exosomes will have a size (average diameter) that is up to 5% of the size of the donor cell. Therefore, especially contemplated exosomes include those that are shed from a cell. As used herein, it is not intended that an extracellular vesicle or exosome of the invention be limited by any particular size or size range.

Exosomes may include any shed membrane bound particle that is derived from either the plasma membrane or an internal membrane. Exosomes can also include cell-derived structures bounded by a lipid bilayer membrane arising from both herniated evagination separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen including tumor-derived microRNAs or intracellular proteins. Exosomes can also include membrane fragments.

Thus, in some embodiments, the disclosure provides a method for purifying or isolating exosomes from a biological sample. The methods may comprise incubating a biological sample with at least one porous three-dimensional device, as disclosed herein, wherein each comprises an exosome-specific binding agent, removing unbound sample, and eluting bound exosomes.

In some embodiments, the devices and methods disclosed herein can purify at least two-fold more exosomes than conventional centrifugation- and microfluidic-based methods. In some embodiments, the device has a maximum exosome binding capacity of greater than 2×10⁸ exosomes per mL. In some embodiments. the maximum exosome binding capacity is greater than about 2×10⁸ exosomes per mL, greater than about 2.5×10⁸ exosomes per mL, greater than about 3×10⁸ exosomes per mL, greater than about 3.5×10⁸ exosomes per mL, greater than about 4×10⁸ exosomes per mL, greater than about 4.5×10⁸ exosomes per mL, or greater than about 5×10⁸ exosomes per mL.

For devices and methods suitable for use in purifying extracellular vesicles and exosomes, the binding agent can be any agent that binds to a fairly ubiquitous surface component of extracellular vesicles or exosomes or a subset of extracellular vesicles or exosomes (e.g., disease or tissue or origin). Suitable surface components include. for example, Alix and Tsg101, tetraspanins (e.g., CD63, CD81, CD9), selectins, integrins, CD40, and other endosome-associated proteins such as Rab GTPases, SNAREs, and flotillin. In some embodiments, cancer-associated surface proteins such as EGFR, EpCAM, KRAS, CD24, CA-125, MUC18, HER2, CD44, Prostate-specific antigen, and the like can be used.

In some embodiments, the binding agent comprises annexin V. When using Annexin V as the binding agent, the binding composition may comprise calcium, as phospholipids (e.g., phosphatidylserine) expressed on the surface of the extracellular vesicles binds to Annexin V in the presence of calcium. Appropriately, the elution buffer may comprise a chelation agent, thereby removing the calcium and disrupting the phosphatidylserine-Annexin V interaction.

Exosomes and extracellular vesicles may be released by mammalian cells for a number of purposes. During pregnancy, for example, exosomes inhibit the production of certain T-cells thereby protecting the fetus. In the case of certain bacterial infections, exosomes derived from infected cells express antigenic fragments of the bacterium to stimulate the immune system against the pathogen. It has been postulated that cancers use the immunomodulatory properties of exosomes in order to evade the immune system. By correlating circulating exosomal markers with molecular characteristics and real-time clinical parameters, the use of circulating exosomes create a “liquid biopsy.” The target cell-derived component may comprise exosomes and extracellular vesicles specific for a disease, disorder or condition.

In some embodiments, the cell-derived component comprises cancer cell-derived exosomes. The cancer cells may be from any cancer, including, but not limited to, breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer, or skin cancer.

The methods may further comprise analyzing or conducting a biological assay with the purified extracellular vesicles or exosomes. In some embodiments, the analysis further comprises quantifying the extracellular vesicles or exosomes in the sample. In some embodiments, the analysis further comprises isolating and analyzing the extracellular vesicles or exosomes for the presence or amount of a biomarker (e.g., DNA or RNA or protein or any combination thereof) or antigen present.

Thus, in some embodiments, the methods may further comprise diagnosing or prognosing a disease or disorder (e.g., cancer) in a subject. In some embodiments, the methods further comprise treating a subject, based on the information obtained. For example, the subject may be administered one or more therapeutic agents (e.g., chemotherapeutic agents) or radiation or may undergo a surgery or other medical procedure.

### 3. Systems or Kits

Also within the scope of the present disclosure are systems or kits that include the disclosed devices or one or more components necessary for making or using the disclosed devices. For example, in some embodiments, the systems or kits include or all of: a porous scaffold, a polymer or polymer precursor, a binding agent, any components necessary for the functionalization of the polymer or conjugation of the binding agent to the polymer, a binding buffer, and an elution buffer.

In some embodiments, the systems or kits may include containers suitable for incubating the sample with the disclosed devices. Each component may be provided in its respective container for use. For example, the systems or kits may comprise one or more (e.g., 2, 5, 10, 15, 20, etc.) of the disclosed devices in a container suitable for use in incubating the sample. In some embodiments, the container suitable for use in incubating the sample comprises vials, bottles, jars, flasks, cell culture devices, flexible bags or other flexible packaging, and the like.

In some embodiments, the systems or kits may further contain materials for procuring or processing the sample.

Individual member components of the systems or kits may be physically packaged together or separately. The components of the systems or kits may be provided in bulk packages (e.g., multi-use packages) or single-use packages. The systems or kits provided herein are in suitable packaging. Suitable packaging includes, but is not limited to, vials, bottles, jars. flexible packaging. and the like.

The systems or kits can also comprise instructions for using the components of the kit. The instructions are relevant materials or methodologies pertaining to the systems or kits. The materials may include any combination of the following: background information, list of components and their availability information (purchase information, etc.), brief or detailed protocols for using the compositions, troubleshooting, references, technical support, and any other related documents. Instructions can be supplied with the systems or kits or as a separate member component, either as a paper form or an electronic form which may be supplied on computer readable memory device or downloaded from an internet website, or as recorded presentation.

It is understood that the disclosed systems or kits can be employed in connection with the disclosed methods.

### 4. Examples

### Materials and Methods

Porous PDMS Preparation The device utilized a sugar cube scaffold as the initial mold for a porous structure. The preparation began with completely covering the 2.05 cm³ sugar cubes with a PDMS precursor and curing agent at a 10:1 ratio. The devices were placed in a vacuum chamber for about an hour to allow for full PDMS penetration into the sugar. PDMS's high resolution allows for penetration on the nanoscale. The PDMS was then set to cure in an oven overnight at about 74° C. The sugar combined PDMS device was removed from the container then the cubes are cut out. They were then placed in an 85° C. water bath for 1 hour to dissolve out the sugar scaffold, replacing the bath if it becomes too saturated. The cubes were set overnight in a 70% ethanol bath to dissolve any remaining sugar. The devices were rung out and dried in preparation for functionalization.

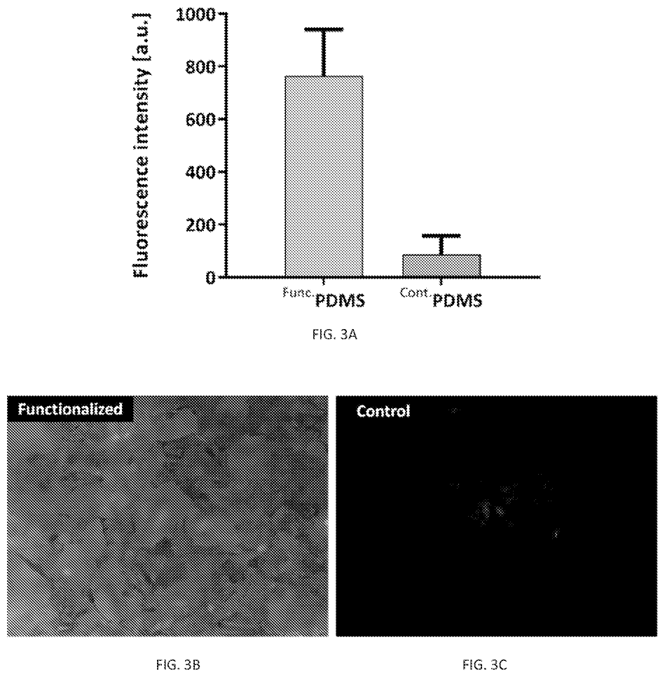

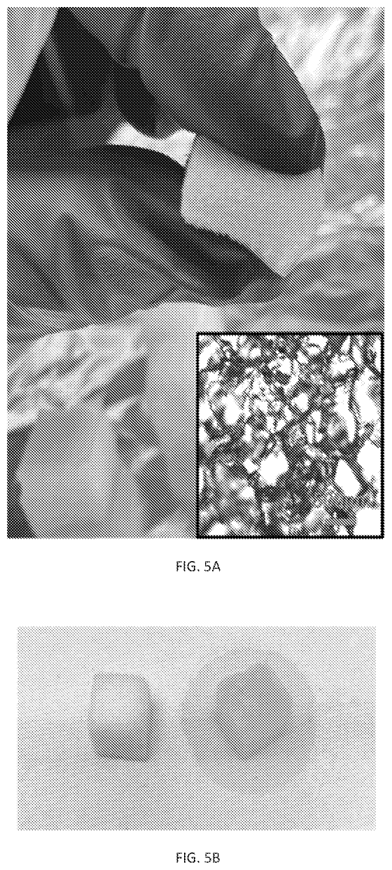

Porous PDMS Functionalization The porous cubes were inherently non-polar due to their PDMS composition. This is ill-suited for the absorption of aqueous solutions such as biological samples. Creating a hydrophilic device required thorough oxidation of the device's large surface area. Due to the denseness of the material, conventional O₂ plasma treatment will not penetrate far enough to be an effective oxidizer. The devices were instead treated in a piranha solution bath containing a mixture of sulfuric acid and hydrogen peroxide making the device's surface hydrophilic (FIG. 4A). The devices were removed after 10 minutes and rinsed carefully with water and ethanol. To enable the material's selective affinity for cancer EVs, the porous PDMS underwent functionalization treatments including silanization, GMBS exposure, and streptavidin conjugation (FIG. 2). These steps were followed by a biotinylated annexin V treatment (Y-T. Kang, et al., Small 15 (2019). 1903600, incorporated herein by reference in its entirety), making the surfaces selective for cancer-derived exosomes.

Exosome Isolation and Release using ^{Porous}ExoChip The porous, high surface area structure of the disclosed device can be used for mid-bulk sample isolation of extracellular vesicles (EVs). EV analysis of large sample sizes is not possible with ultracentrifugation (˜40 ml) or previous microfluidic devices (<1 ml), largely due to the limited volume of the capsules in ultracentrifugation high-speed rotation. However, ^{Porous}ExoChip's immune-based separation is not limited by similar physical constraints. Once fully functionalized, the devices can be introduced to sample sizes on the milliliter scale making for quicker EV isolation.

After full functionalization, the device was introduced to biological samples containing cancer-derived exosomes along with a binding buffer that contains calcium ions. The devices were then incubated in 50 mL sealed tubes and set on a rocker for a 40-minute incubation time. The devices were removed and placed onto washing plates and rinsed with binding buffer. They were then subjected to EDTA treatment to release the captured exosomes by disrupting the affinity between the surface protein and the Annexin V conjugated PDMS.

### Example 1

### Porous PDMS

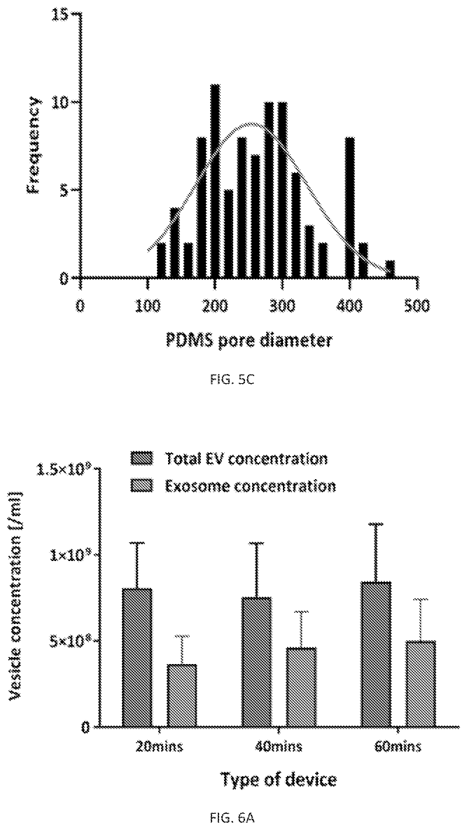

Optimization experiments were run comparing different device cube sizes and their relative interaction with EVs. These tests demonstrated that the smaller cubes—with a larger relative surface area—were more effective at exosome isolation. However, the additional steps and increased experimental user difficulty ultimately outweighed the smaller cube's superior isolation. The optimized device size had dimensions close to the sugar cube that was used as a scaffold. It possessed a volume of about 2.05 cm³ and the oxidized device held an aqueous volume of about 1.4 mL (FIG. 5B). These relatively large volumes allowed for bulk analysis. After microscopic analysis. the average pore size was about 270 microns with pores ranging from 100 to 450 microns (FIGS. 5A and 5C). The average outer surface pore size was smaller than the middle layers of the device.

### Example 2

### Isolation of Exosomes using ^{Porous}ExoChip

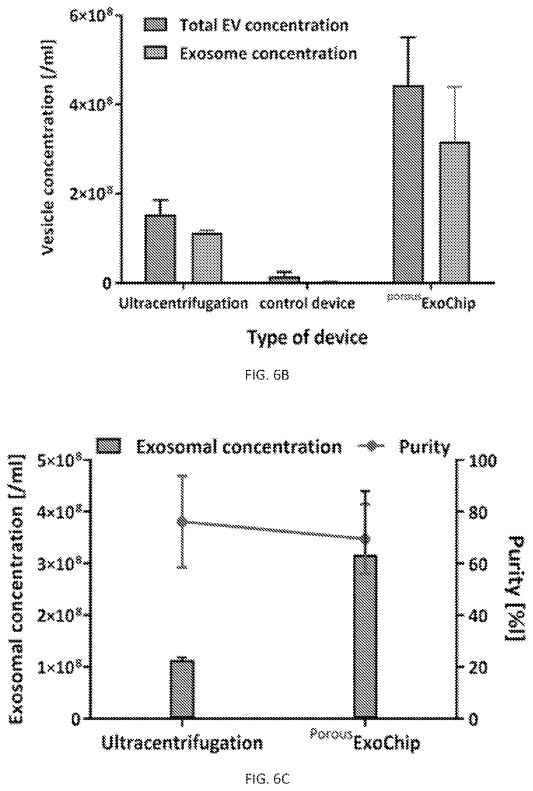

The exosome isolation utilized an immune affinity interaction between the Annexin V treated PDMS and cancer-derived exosomes. This occurred because the phosphatidylserine protein that is expressed on the surface of the EVs will bind to the device when in the presence of a binding buffer (BB). The BB introduces calcium ions necessary for this interaction (FIG. 4B). During optimization of the exosome isolation procedure, the number of devices, incubation time, and incubation environment were tested. The optimal number of cube devices per volume of biological sample was determined to be a ratio of 1 device per 2.5 mL of sample. The incubation time displayed an effect on the exosome uptake, but an incubation time of 40 minutes was found to be the most practical (FIG. 6A). Lastly, through experimentation of the incubation environment, a simple rocker setup was preferential in comparison to a centrifugation treatment in regards to sample penetration into the device.

While conventional exosome isolations in biological samples have had success, they require expensive equipment that must be operated in a controlled lab environment. However, the disclosed PDMS device can be functionalized to also be an effective means of EV isolation and even outperform these traditional procedures, at much lower cost. Comparing the disclosed porous device to previous isolation methods such as ultracentrifugation, the ^{Porous}ExoChip demonstrates superior exosomal isolation.

The ^{Porous}ExoChip measured exosome concentrations double that of ultracentrifugation. The total EV concentration for the disclosed device was approximately 4.3×10⁸ exosomes per mL while ultracentrifugation yielded only about 1.8×10⁸ exosomes per mL. In addition to its superior EV yield, there was no significant difference in the isolated EV sample's purity (% of exosome-sized vesicles in the sample) when compared to ultracentrifugation. Both had median purity percentages above 70% (FIGS. 6B and 6C).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

what is claimed is:

1. A method for purifying or isolating a target cell-derived component comprising:
incubating a sample with at least one porous three-dimensional device, wherein each device comprises a polymer-based surface conjugated with a binding agent for the target cell-derived component;
removing unbound sample; and
eluting the target cell-derived component with an elution buffer composition configured to disrupt target cell-derived component and binding agent interaction.

2. The method of claim 1, wherein the method does not comprise centrifugation.

3. The method of claim 1 or 2, wherein the at least one porous three-dimensional device is a cube, cuboid, pyramid, prism, or polyhedron.

4. The method of any of claims 1-3, wherein the at least one porous three-dimensional device comprise pores from 100 to 450 microns.

5. The method of any of claims 1-4, wherein the at least one porous three-dimensional device has a maximum accommodated aqueous volume of at least 0.5 mLs.

6. The method of any of claims 1-5, wherein the polymer comprises polydimethylsiloxane (PDMS).

7. The method of any of claims 1-6, wherein the incubating comprises fully or partially submerging the at least one porous three-dimensional device in the sample for a period of time.

8. The method of claim 7, wherein the incubating further comprises agitating the fully or partially submerged at least one porous three-dimensional device in the sample.

9. The method of any of claims 1-8, wherein the incubating further comprises adding binding composition which facilitates target cell-derived component and binding agent interaction.

10. The method of any of claims 1-9, wherein the sample is a biological sample.

11. The method of wherein the biological sample is obtained from a subject.

12. The method of claim 11, wherein the subject has or is suspected of having a disease or disorder.

13. The method of any of claims 1-12, wherein the sample has a volume of at least 1 mL.

14. The method of any of claims 1-13, wherein the sample has a volume of at least 50 mLs.

15. The method of any of claims 1-14, wherein the target cell-derived component comprises cell fragments.

16. The method of any of claims 1-14, wherein the target cell-derived component comprises a biomolecule.

17. The method of any of claims 1-14, wherein the target cell-derived component comprises extracellular vesicles.

18. The method of any of claims 1-14, wherein the target cell-derived component comprises exosomes.

19. The method of claim 17 or claim 18, wherein the target cell-derived component comprises cancer cell-derived exosomes.

20. The method of any of claims 17-19, wherein the binding agent comprises annexin V.

21. The method of claim 20, wherein the binding composition comprises calcium.

22. The method of any of claims 17-21, wherein the at least one porous three-dimensional device has a maximum exosome binding capacity of greater than 2×10⁸ exosomes per mL.

23. The method of any of claims 1-22, further comprising analyzing the target cell-derived component.

24. The method of claim 23, wherein the analyzing comprises conducting a biological assay on the target cell-derived component.

25. A three-dimensional porous device comprising a polymer-based surface conjugated with a binding agent.

26. The device of claim 25, wherein the device is a porous cube.

27. The device of claim 25 or 26, wherein the polymer comprises polydimethylsiloxane (PDMS).

28. The device of claim 27, wherein the PDMS is oxidized and hydrophilic.

29. The device of any of claims 25-28, wherein the device has a maximum accommodated aqueous volume of 1.0-2.0 mLs.

30. The device of any of claims 25-29, wherein the binding agent specifically binds a target cell-derived component.

31. The device of claim 30, wherein the target cell-derived component is an exosome.

32. A method for fabricating a porous three-dimensional device comprising:
providing a three dimensional porous scaffold, wherein the scaffold is configured to be removed;
covering the three dimensional porous substrate with a polymer or polymer precursor;
optionally, curing or crosslinking the polymer or polymer precursor; and
removing the scaffold.

33. The method of claim 32, wherein the porous scaffold is a sugar cube.

34. The method of claim 32 or 33, wherein the polymer comprises polydimethylsiloxane (PDMS).

35. The method of any of claims 32-34, further comprising rendering the polymer surface hydrophilic.

36. The method of claim 35, wherein the rendering the polymer surface hydrophilic comprises oxidation.

37. The method of any of claims 32-36, further comprising functionalization of the polymer with a binding agent.

38. The method of claim 37, wherein the binding agent specifically binds a target cell-derived component.

39. The method of claim 38, wherein the target cell-derived component is an exosome.

40. A method purifying or isolating a target cell-derived component comprising:
incubating at least one porous three-dimensional device of any of claims 25-31 or at least one porous three-dimensional device made by a method of any of claims 31-39 with a sample;
removing unbound sample; and
eluting the target cell-derived component.

41. A system or kit comprising a porous three-dimensional device of any of claims 25-31 or components necessary for making thereof.

42. A system or kit comprising one or more of a porous three-dimensional device of any of claims 25-31 in a container suitable for use in incubating a sample.

43. Use of a porous three-dimensional device of any of claims 25-31 or a porous three-dimensional device made by a method of any of claims 31-39 for purifying or isolating a target cell-derived component.
